(19) United States
(12) Patent Application Publication
(10) Pub. No.: US 2025/0177267 A1
(43) Pub. Date: Jun. 5, 2025

(54) A CLEANSING COMPOSITION

(71) Applicant: Conopco, Inc., d/b/a UNILEVER, Englewood Cliffs, NJ (US)

(72) Inventors: Sameer Keshav BARNE, Bangalore (IN); Maya Treesa SAJI, Bangalore (IN)

(73) Assignee: Conopco, Inc., d/b/a UNILEVER, Englewood Cliffs, NJ (US)

(21) Appl. No.: 18/845,471

(22) PCT Filed: Mar. 8, 2023
(86) PCT No.: PCT/EP2023/055829

§ 371 Date: Sep. 10, 2024

(30) Foreign Application Priority Data
Mar. 16, 2022 (EP) 22162395.2

(51) Int. Cl. A61K 8/368; A61K 8/20; A61K 8/23; A61K 8/365; A61K 8/44; A61K 8/46; A61L 2/00; A61L 101/36; A61Q 19/10

(52) U.S. Cl. CPC A61K 8/368; A61K 8/20; A61K 8/23; A61K 8/365; A61K 8/442; A61K 8/463; A61L 2/0088; A61Q 19/10; A61L 2101/36

(57) ABSTRACT

The present invention relates to a cleansing composition. Particularly, the present invention relates to a cleansing composition that provides superior antimicrobial e.g. antibacterial, effect in consumer relevant contact time of e.g. less than 5 minutes e.g 10 to 30 sec as well as required viscosity.

### FIELD OF THE INVENTION

The present invention relates to a cleansing composition. Particularly, the present invention relates to a cleansing composition that provides superior antimicrobial e.g. antibacterial, effect.

### BACKGROUND OF THE INVENTION

WO2018 121946 (Unilever) discloses an antimicrobial composition at the pH of skin. The composition comprises (a) 5 to 40% by weight of an ammonium salt having at least one covalently bonded hydrogen attached to the nitrogen or of an anionic surfactant with ammonium group as counter ion or mixtures thereof; and, (b) 0.1 to 20% by weight of a carboxylic acid with pKa value greater than 4.5.

However, the carboxylic acids discussed in the prior art are those with pKa greater than 4.5 e.g. hexanoic acid (pKa=4.72) and octanoic acid (pKa=4.89). Although they provide good antimicrobial efficacy in presence of anionic surfactants system as discussed therein, these acids are medium chain fatty acids that are costly and have a peculiar odor associated with them which is less preferred. If such acids are used, the odor then needs to be tackled with fragrance systems which further increases the costs.

On the other hand, carboxylic acids e.g. aromatic carboxylic acids such as benzoic acid and cinnamic acid may be used in cleansing compositions. However, using these acids affects viscosity of a cleansing composition.

Need therefore exists to provide a cleansing composition that overcomes one or more drawbacks associated with prior art.

It has now been found that addition of ammonium salts e.g. ammonium chloride, significantly improves the antimicrobial efficacy of aromatic carboxylic acids e.g. benzoic acid, which are easier to incorporate in cleansing compositions without giving rise to odor issues. Moreover, the antimicrobial effect is found to be obtained in short duration e.g. 10 to 30 seconds, e.g. 20 seconds. It has also been found that addition of ammonium salts also provided required viscosity to the cleansing composition despite the composition contained aromatic carboxylic acids e.g. benzoic acid.

### SUMMARY OF THE INVENTION

In a first aspect, the present invention relates to a cleansing composition comprising:
a. from 0.1 to 10 wt % ammonium salt selected from ammonium chloride, ammonium benzoate, ammonium citrate, ammonium carbonate, ammonium acetate, ammonium sulphate, isopropyl ammonium chloride and mixtures thereof; and
b. from 0.01 to 10 wt % aromatic carboxylic acid selected from benzoic acid, 2-hydroxybenzoic acid, 3-hydroxybenzoic acid, 4-hydroxybenzoic acid, cinnamic acid and mixtures thereof, wherein the composition has pH in the range 2.5 to 4.5.

In a second aspect, the present invention relates to a method of providing antimicrobial effect comprising the steps:
i. applying the composition of the first aspect on to a surface; and
ii. optionally, rinsing the surface on to which the composition is applied.

In a third aspect, the present invention relates to use of the composition of the first aspect for providing antimicrobial effect.

In a fourth aspect, the present invention relates to use of an ammonium salt as an enhancer of the antimicrobial efficacy of a cleansing composition, said cleansing composition comprising aromatic carboxylic acid selected from benzoic acid, 2-hydroxy benzoic acid, 3-hydroxybenzoic acid, 4-hydroxybenzoic acid, cinnamic acid and mixtures thereof.

In a fifth aspect, the present invention relates to use of ammonium salts of the first aspect in a liquid cleansing composition as a viscosity modifying agent.

### DETAILED DESCRIPTION OF THE INVENTION

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “x to y” are understood to include x and y. When for a specific feature multiple preferred ranges are described in the format “x to y”, it is understood that all ranges combining the different endpoints are also contemplated. Unless specified otherwise, amounts as used herein are expressed in percentage by weight based on total weight of the composition and is abbreviated as “wt %”. The use of any and all examples or exemplary language e.g. “such as” provided herein is intended merely to better illuminate the invention and does not in any way limit the scope of the invention otherwise claimed.

For the purpose of the present invention, the terms ‘antimicrobial benefit’ or ‘antimicrobial effect’ or ‘antimicrobial efficacy’, may be used interchangeably and they denote the same meaning. Preferably, ‘antimicrobial benefit’ or ‘antimicrobial effect’ or ‘antimicrobial efficacy’ means reduction in number of microbes e.g. bacteria, fungi, viruses. Such antimicrobial effect is obtained when the composition of the present invention is preferably applied on to a surface that may be an inanimate or animate surface. More preferably, the composition is applied to an animate substrate e.g. human skin. ‘Skin’ as used herein, preferably means to include skin on any part of the body e.g. face, neck, chest, back, arms, underarms, hands, legs, buttocks and scalp.

In a first aspect, the present invention relates to a cleansing composition comprising:
a. from 0.1 to 10 wt % ammonium salt selected from ammonium chloride, ammonium benzoate, ammonium citrate, ammonium carbonate, ammonium acetate, ammonium sulphate, isopropyl ammonium chloride and mixtures thereof; and
b. from 0.01 to 10 wt % aromatic carboxylic acid selected from benzoic acid, 2-hydroxy benzoic acid, 3-hydroxybenzoic acid, 4-hydroxybenzoic acid, cinnamic acid and mixtures thereof wherein the composition has pH in the range 2.5 to 4.5.

### Ammonium Salts

Generally, salts are added to cleansing compositions as electrolytes to provide the right ionic strength. The addition of salts to the aqueous solution of the surfactants changes the solution properties, such as the critical micellar concentration, as well as the phase behaviors of the surfactant.

A cleansing composition according to the of the present invention (the composition) comprises ammonium salts selected from ammonium chloride, ammonium benzoate, ammonium citrate, ammonium carbonate, ammonium acetate, ammonium sulphate, isopropyl ammonium chloride or mixtures thereof.

Preferably, the ammonium salts are selected from ammonium chloride, ammonium citrate, ammonium sulfate and mixtures thereof. More preferably, the ammonium salt selected is ammonium chloride.

The composition comprises from 0.1 to 10 wt %, preferably from 0.5 to 9 wt %, more preferably from 1 to 8 wt %, further more preferably from 1 to 7 wt %, even more preferably from 1 to 6 wt %, still more preferably from 1 to 5 wt %, yet more preferably from 1 to 4 wt %, still further more preferably from 1 to 3 wt % and yet further more preferably from 1 to 2 wt % of the ammonium salts described above.

It has been found that the ammonium salts provide a synergistic antimicrobial effect when used in combination with the aromatic carboxylic acids described below. A new use of the ammonium salts has also been found that when used in a liquid cleansing composition, they provide the required viscosity. Preferably, viscosity is in the range from 2000 to 6000 cps at 25° C.

### Aromatic Carboxylic Acids

The composition also comprises an aromatic carboxylic acid selected from benzoic acid, 2-hydroxy benzoic (also known as salicylic acid) acid, 3-hydroxybenzoic acid (m-salicylic acid), 4-hydroxybenzoic acid, cinnamic acid and mixtures thereof.

Preferably, the aromatic carboxylic acids are selected from benzoic acid, 2-hydroxy benzoic acid, 3-hydroxybenzoic acid and mixtures thereof. More preferably, the aromatic acid selected is benzoic acid.

The composition comprises the aromatic carboxylic acid from 0.01 to 10 wt %, preferably from 0.1 to 9 wt %, more preferably from 0.2 to 8 wt %, even more preferably from 0.5 to 7 wt %, further more preferably from 1 to 6 wt %, still more preferably from 2 to 5 wt % and yet more preferably from 3 to 4 wt %.

It has been found that the ammonium salt as described above in combination with the carboxylic acids described above, provides synergistic antimicrobial effect.

### Anionic Surfactants:

Preferably, the composition further comprises an anionic surfactant. Preferably, the anionic surfactant is selected from alkyl ether sulfates (AES), alkyl sulfosuccinate and mixtures thereof.

AES are anionic surfactants of the general formula: R₁—(OR′)ₙ—O—SO₃⁻M⁺, wherein:
R₁ is saturated or unsaturated C₈-C₁₆, preferably C₁₂-C₁₄ alkyl chain; preferably, R₁ is a saturated C₈-C₁₆, more preferably a saturated C₁₂-C₁₄ alkyl chain;
R′ is ethylene; n is from 1 to 18; preferably from 1 to 15, more preferably from 1 to 10 and even more preferably from 1 to 5,
M⁺ is a suitable cation which provides charge neutrality, preferably sodium, calcium, potassium, or magnesium, more preferably a sodium cation.

Examples of AES that may be used as anionic surfactant in the composition include sodium lauryl ether sulfate (SLES), sodium myristyl ether sulfate and sodium palmityl ether sulfate and mixtures thereof. Preferred AES is SLES having 1 to 3 ethylene oxide units per molecule. SLES having 1 to 2 ethylene oxide units per molecule is more preferred.

Examples of alkyl sulfosuccinate that may be used as an anionic surfactant includes disodium lauryl sulfosuccinate.

Preferably, the composition comprises from 2 to 40 wt %, more preferably from 5 to 35 wt %, even more preferably from 7 to 30 wt %, still more preferably from 9 to 20 wt % and further more preferably from 10 to 15 wt % anionic surfactants.

### Amphoteric Surfactants

Preferably, the composition further comprises amphoteric surfactants as a co-surfactant. They provide foam boost and improve sensorial of the composition. Preferably, amphoteric surfactants are selected from cocamidopropyl betaine (CAPB), cocamide monoethanolamide (CMEA), cocoamphoacetate and mixtures thereof.

Preferably, the composition comprises from 0.1 to 10 wt %, more preferably from 0.5 to 8 wt %, even more preferably from 1 to 7 wt %, further more preferably from 2 to 5 wt % and still more preferably from 3 to 5 wt % amphoteric surfactants.

### Essential Oils

Preferably the composition further comprises one or more essential oils. These essential oils may be selected from thymol, terpineol, carvacrol, eugenol, menthol and mixtures thereof. More preferably, the essential oils may be selected from thymol, terpineol, carvacrol, eugenol and mixtures thereof. Even more preferably, the essential oils may be selected from thymol, terpineol, eugenol and mixtures thereof. Further more preferably, the essential oils may be selected from thymol, terpineol and mixtures thereof.

Preferably, the composition comprises from 0.0001 to 1 wt %, more preferably 0.001 to 0.5 wt %, even more preferably from 0.01 to 0.25 wt % and further more preferably from 0.01 to 0.1 wt % of one or more of these essential oils.

### PH of the Composition

The pH of the composition is in the range from 2.5 to 4.5 more preferably, the pH of the composition is in the range from 3.0 to 4.5. Even more preferably, the pH of the composition is in the range from 3.5 to 4.5 and further more preferably from 4.0 to 4.5.

### Format of the Composition:

Preferably, the composition is in liquid form e.g. a liquid handwash composition, a liquid bodywash composition. Preferably, the liquid cleansing composition has viscosity in the range from 2000 to 6000, more preferably from 2500 to 5500, even more preferably from 3000 to 5000, further more preferably from 3500 to 4500 and still more preferably from 3500 to 4000 cP at 25° C.

Alternatively, the composition may be in the form of a spray.

Preferably, the composition further comprises water soluble/dispersible polymers. These polymers can be cationic, anionic, amphoteric or nonionic types with molecular weights higher than 100,000 Dalton. They are known to increase the viscosity and stability of liquid cleansing compositions, to enhance in-use and after-use skin sensory feels, and to enhance lather creaminess and lather stability. Such polymers may preferably be used in amounts from 0.1 to 10 wt %, more preferably from 0.1 to 5 wt %, even more preferably from 0.25 to 3 wt % and further more preferably from 0.5 to 2 wt %

Examples of water soluble/or dispersible polymers include the carbohydrate gums such as cellulose gum, microcrystalline cellulose, cellulose gel, hydroxyethyl cellulose, hydroxypropyl cellulose, hydroxypropyl methyl cellulose, sodium carboxymethylcellulose, methyl cellulose, ethyl cellulose, guar gum, gum karaya, gum tragacanth, gum arabic, gum acacia, gum agar, xanthan gum and mixtures thereof; modified and nonmodified starch granules and pregelatinized cold water soluble starch; emulsion polymers such as Aculyn® 28, Aculyn® 22 or Carbopol® Aqua SF1; cationic polymer such as modified polysaccharides including cationic guar available from Rhone Poulenc under the trade name Jaguar® C13S, Jaguar® C14S, Jaguar® C17, or Jaguar® C16; cationic modified cellulose such as UCARE® Polymer JR 30 or JR 40 from Amerchol; N-Hance® 3000, N-Hance® 3196, N-Hance® GPX 215 or N-Hance® GPX 196 from Hercules; synthetic cationic polymer such as Merquat® 100, Merquat® 280, Merquat® 281 and Merquat® 550 sold by Nalco; cationic starches such as StaLok® 100, 200, 300 and 400 sold by Staley Inc.; cationic galactomannans such as Galactasol® 800 series by Henkel, Inc.; Quadrosoft® LM-200; and Polyquaternium-24©. Also suitable are high molecular weight polyethylene glycols such as Polyox® WSR-205 (PEG 14M), Polyox® WSR—N-60K (PEG 45), and Polyox® WSR-301 (PEG 90M).

Preferably, the composition further comprises polymers selected from vinyl Pyrrolidone/vinyl acetate copolymer.

Preferably, the composition comprises water from 60 to 98 wt %, more preferably from 65 to 95 wt %, even more preferably from 70 to 90 wt %, further more preferably from 75 to 85 wt % and still more preferably from 80 to 85 wt % water.

Preferably, the composition further comprises one or more skin lightening agents. Preferably, the skin lightening agent may be selected from aloe extract, ammonium lactate, arbutin, azelaic acid, kojic acid, butyl hydroxy anisole, butyl hydroxy toluene, citrate esters, 3 diphenyl propane derivatives, ellagic acid, fennel extract, gluco pyranosyl-1-ascorbate, gluconic acid, glycolic acid, hydroquinone, 4 hydroxyanisole, linoleic acid, magnesium ascorbyl phosphate, 2,4 resorcinol derivatives, 3,5 resorcinol derivatives, vitamins like vitamin B3, vitamin B6, vitamin B12, vitamin C, vitamin A, resorcinol derivatives and mixtures thereof.

Preferably, the composition further comprises preservatives to protect the composition against the growth of potentially harmful microorganisms. Particularly preferred preservatives include hydantoin derivatives, propionate salts, a variety of quaternary ammonium compounds, phenoxyethanol, imidazolidinyl urea, sodium dehydroacetate and benzyl alcohol. Preservatives are preferably used in amounts ranging from 0.01 wt % to 2 wt % and more preferably from 0.1 to 1 wt %.

The composition may further comprise emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

The compositions may further comprise a wide range of other optional components e.g. antioxidants, binders, biological additives, buffering agents, colorants, astringents, fragrance, humectants, opacifying agents, conditioners, pH adjusters, skin soothing agents and skin healing agents.

In a second aspect, the invention relates to a method of providing antimicrobial effect comprising the steps:
a. applying the composition of the first aspect on to a surface; and
b. optionally, rinsing the surface on to which the composition is applied.

An antimicrobial effect is obtained when the composition of the present invention is applied on to a surface that may be an inanimate e.g. hard surfaces; or an animate surface e.g. human skin. More preferably, the composition is applied on to an animate surface e.g. human skin. ‘Skin’ as used herein, preferably means to include skin on any part of the body e.g. face, neck, chest, back, arms, underarms, hands, legs, buttocks and scalp.

Preferably, in step b, if the surface on to which the composition is applied is rinsed with water, then rinsing is carried out in less than 5 minutes, more preferably in less than 4 minutes, even more preferably in less than 3 minutes, further more preferably in less than 1 minute e.g. 30 or 20 seconds, after the step of applying the composition on to a surface.

Preferably, the method is non-therapeutic and/or cosmetic in nature.

In a third aspect, the present invention also relates to use of a composition of the first aspect for providing antimicrobial benefit. Preferably, the use is non-therapeutic and/or cosmetic in nature.

In a fourth aspect, the present invention relates to use of an ammonium salt as an enhancer of the antimicrobial efficacy of a cleansing composition, said cleansing composition comprising aromatic carboxylic acid selected from benzoic acid, 2-hydroxy benzoic acid, 3-hydroxybenzoic acid, 4-hydroxybenzoic acid, cinnamic acid and mixtures thereof. Preferably, the use is non-therapeutic and/or cosmetic in nature.

In a fifth aspect, the present invention also relates to use of the ammonium salts described hereinabove, in a liquid cleansing composition as a viscosity modifying agent. Preferably, the viscosity is in the range from 2000 to 6000 cP at 25° C. It has been found that ammonium salts e.g. ammonium chloride, ammonium benzoate, ammonium citrate, ammonium carbonate, ammonium acetate, ammonium sulphate, isopropyl ammonium chloride, aided to obtain desired viscosity for a liquid cleansing composition. Preferred ammonium salts are ammonium chloride, ammonium citrate, ammonium sulphate and mixtures thereof. Preferably, the viscosity of the liquid composition in in the range from 2000 to 6000 cP at 25° C.

The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

The invention is now further illustrated through the following non-limiting examples.

### Examples

### Protocols

The following protocol was used to evaluate biocidal activity.

### IN-VITRO TIME-KILL PROTOCOL—ASTM 2783

### Preparation of the Bacterial Culture:

For these experiments, Escherichia.coli ATCC 10536 was used in the study, which represents gram-negative bacteria. The bacteria were grown overnight on Tryptic soya agar (TSA) plate. The bacterial cell density was then adjusted at 620 nm to a pre-calibrated optical density to get the final count of 10⁹ cfu/mL in saline (0.86% NaCl) by using a spectrophotometer.

### Assay Protocol:

9.9 mL of the composition of different examples (as stated above) was taken in different sample containers to each of those container 0.1 mL of bacterial culture was added just before performing the assay and mixed well to obtain a mixture. A timer was started immediately after the addition of the culture. The mixture was kept for a specific contact for a duration ranging from 10 seconds to 30 seconds e.g. 20 seconds.

At the end of the each contact time, the antibacterial activity of the samples was neutralized immediately, by addition of 1 mL each of the above mixture to 9 mL of an appropriate neutralizing broth which is validated for the test system. The neutralized samples were then serially diluted up to 5 dilution in neutralizer broth and plated on TSA (40 gpL—Difco) in duplicates.

The log reduction was calculated by comparing with the bacterial control. The bacterial control used for this purpose was a mixture prepared by addition of 0.1 mL of bacterial culture to 9.9 mL of saline; the mixture was then serially diluted and plated on TSA. After solidification of the TSA plates, the plates were incubated at 37° C. for 48 hours. The colonies on the plates were counted.

Log reduction greater than 5 means 99.99% reduction in the number of CFU and log reduction less than 0.5 means no reduction in the number of CFU that means no antimicrobial efficacy. Log reduction greater than 5 also denotes complete kill.

### Measurement of Viscosity

Zero calibration was done every time when using the viscometer (calibration without any spindle). The spindle RV-5 was fixed in the viscometer. The RPM was set to 20 and measurement time was set to 30 sec. The spindle was submerged into a test sample (a composition) until the mark mentioned on the spindle. As the measurement was started, the spindle rotated inside the sample and corresponding torque was measured and the instrument showed the calculated viscosity. The viscosity was measured after 30 sec; and was measured at room temperature, 25° C. For this, Brookfield RV viscometer with RV-5 spindle, at 20 RPM for 30 sec was used.

### Preparation of Compositions Shown in Tables Below:

Briefly, the compositions as shown in tables below were prepared by mixing the surfactants in water at 70 to 75° C. followed by addition of salts and carboxylic acids; and optionally a polymer, at 35 to 45° C. e.g. 40° C. Minors like perfume, if any, were added after the compositions were cooled to room temperature e.g. 25° C.

The compositions as shown in table 1 below were prepared and their antimicrobial efficacy was evaluated as per the protocol described above.

TABLE 1

| | A | B | C | 1 | 2 |
|---|---|---|---|---|---|
| Example | (wt %) | (wt %) | (wt %) | (wt %) | (wt %) |
| SLES.1 EO | 6.3 | 6.3 | 6.3 | 6.3 | 6.3 |
| CAPB | 1.5 | 1.5 | 1.5 | 1.5 | 1.5 |
| Ammonium chloride | — | — | 0.5 | 0.5 | 1 |
| Sodium chloride | 0.5 | 0.5 | 0.5 | 0.5 | 0.5 |
| Benzoic acid | — | 2 | — | 2 | 2 |
| Glycerin | 0.1 | 0.1 | 0.1 | 0.1 | 0.1 |
| Minors: perfume, | 2 | 2 | 2 | 2 | 2 |
| polymers and dyes | | | | | |
| Water | To 100 | To 100 | To 100 | To 100 | To 100 |
| Antimicrobial effect: | | | | | |
| Log reduction | 0.2 ± 0.035 | 2.2 ± 0.183 | <0.5 | 4.9 ± 0.141 | >5 |
| of E. coli | | | | | |
| (ATCC 10536), | | | | | |
| 20 seconds | | | | | |

pH = 4.2

Viscosity of composition of example 1 was found to be 2380 cps at 25° C. when measured as described above.

The data in table 1 shows synergistic antimicrobial effect obtained when ammonium salt and an aromatic carboxylic acid according to the present invention were used.

Further, antimicrobial efficacy of commercially available liquid handwash products was evaluated using the same protocol described above; and the results were as shown in table 2 below:

TABLE 2

| | Commercially |
|---|---|
| | available |
| Example | product |
| D | Dettol |
| | Original HW |
| Antimicrobial Effect: | |
| Log reduction E. coli | 3.0 |
| (ATCC 10536), | |
| 20 seconds | |

The composition as per the present invention provided superior antimicrobial effect than obtained using commercially available products.

Further, compositions shown in examples 3 to 8 as per the present invention and a comparative example, example E, were prepared and antimicrobial efficacy was measured as described earlier.

TABLE 3

| | Example | | | | | | |
|---|---|---|---|---|---|---|---|
| | E | 3 | 4 | 5 | 6 | 7 | 8 |
| | (wt %) | (wt %) | (wt %) | (wt %) | (wt %) | (wt %) | (wt %) |
| SLES.1EO | 6.3 | 6.3 | 6.3 | 6.3 | 6.3 | 6.3 | 6.3 |
| CAPB | 1.5 | 1.5 | 1.5 | 1.5 | 1.5 | 1.5 | 1.5 |
| Ammonium chloride | — | 0.5 | — | — | 0.5 | — | — |
| Ammonium citrate | — | — | 0.5 | — | — | 0.5 | — |
| Ammonium sulfate | — | — | — | 0.5 | — | — | 0.5 |
| Sodium chloride | 0.5 | — | — | — | — | — | — |
| Salicylic acid | 2 | — | — | — | 2 | 2 | 2 |
| Benzoic acid | — | 2 | 2 | 2 | — | — | — |
| Water | To | To | To | To | To | To | To |
| | 100 | 100 | 100 | 100 | 100 | 100 | 100 |
| Antimicrobial effect: | | | | | | | |
| Log reduction of E. | 1.7 ± 0.29 | >5 | >5 | >5 | 2.8 ± 0.082 | >5 | >5 |
| coli (ATCC 10536), | | | | | | | |
| 20 seconds | | | | | | | |

pH = 4 ± 0.1

The data in table 3 above shows the antimicrobial effect obtained when ammonium salts as per the present invention and carboxylic acids as per the present invention, were used in combination with each other. The data also shows the specificity of ammonium salts as the composition of example E made with sodium chloride, a salt not according to the present invention, did not show comparable antimicrobial effect as that shown by other inventive examples.

Further, compositions as shown in examples F and 9 were prepared; and viscosity was measured as described above.

TABLE 4

| | F | 9 |
|---|---|---|
| Example | (wt %) | (wt %) |
| SLES.1EO | 6.30 | 6.30 |
| CAPB | 1.50 | 1.50 |
| Ammonium Chloride | — | 1 |
| Sodium Chloride | 1 | — |
| Benzoic acid | 2 | 2 |
| Water | To 100 | To 100 |
| Viscosity (cP) at 25° C. | 1800 | 3100 |

pH = 4.5

The data in table 4 above shows that the composition of example 9 that contained an ammonium salt e.g. ammonium chloride, exhibited enhanced viscosity as compared to that exhibited by example F.

1. A cleansing composition comprising:
a. from 0.1 to 10 wt % ammonium salt selected from ammonium chloride, ammonium benzoate, ammonium citrate, ammonium carbonate, ammonium acetate, ammonium sulphate, isopropyl ammonium chloride and mixtures thereof; and
b. from 0.01 to 10 wt % aromatic carboxylic acid selected from benzoic acid, 2-hydroxy benzoic acid, 3-hydroxybenzoic acid, 4-hydroxybenzoic acid, cinnamic acid and mixtures thereof;
wherein the composition has a pH in the range of 2.5 to 4.5.

2. The cleansing composition according to claim 1, wherein the ammonium salt is ammonium chloride.

3. The cleansing composition according to claim 1, wherein the carboxylic acid is selected from benzoic acid, 2-hydroxybenzoic acid, 3-hydroxybenzoic acid and mixtures thereof.

4. The cleansing composition according to claim 1, wherein the carboxylic acid is benzoic acid.

5. The cleansing composition according to claim 1, wherein the composition further comprises from 2 to 40 wt % anionic surfactant selected from alkyl ether sulfate, alkyl sulfosuccinate and mixtures thereof.

6. The cleansing composition according to claim 1, wherein the composition further comprises 0.1 to 10 wt % of an amphoteric surfactant selected from cocamidopropyl betaine, cocamide monoethanolamide, cocoamphoacetate and mixtures thereof.

7. The cleansing composition according to claim 1, wherein the composition further comprises an essential oil selected from thymol, terpineol, carvacrol, eugenol, menthol and mixtures thereof.

8. The cleansing composition according to claim 1, wherein the composition is a liquid cleansing composition.

9. The cleansing composition according to claim 1, wherein a viscosity of the composition is in the range of 2000 to 6000 cP at 25° C.

10. The cleansing composition according to claim 1, wherein the composition is in the form of a spray.

11. A method of providing antimicrobial effect comprising the steps:
a. applying the cleansing composition according to claim 1 on to a surface; and
b. optionally, rinsing the surface on to which the composition is applied.

12. The method as claimed in claim 11, wherein the step of rinsing the surface is carried out within 5 minutes from applying the cleansing composition to the surface.

13. (canceled)

14. (canceled)
